(19) United States
(12) Patent Application Publication
(10) Pub. No.: US 2006/0262271 A1
(43) Pub. Date: Nov. 23, 2006

(54) Keratometric module for coupling to slit lamps and or ocular microscopes

(76) Inventors: Lillane Ventura Schiabel, Sao Paulo (BR); Jean-Jacques Georges Soares de Groote, Sao Carlos (BR)

Correspondence Address: KILE GOEKJIAN REED & MCMANUS, 1200 NEW HAMPSHIRE AVE, NW, SUITE 570, WASHINGTON, DC, 20036, US

(21) Appl. No.: 10/530,405

(22) PCT Filed: Dec. 19, 2003
(86) PCT No.: PCT/BR03/00200

§ 371 Date: Apr. 7, 2005

(30) Foreign Application Priority Data
Jul. 7, 2003 (BR) 003038

(51) Int. Cl. A61B 3/10

(52) U.S. Cl. 351/212

(57) ABSTRACT

The present invention refers to an ophthalmic instrument coupled to a Slit Lamp or Ocular Microscope (1) that utilizes imaging to selectively measure corneal curvature and examine its surface under examination mode of the instrument in reference, constituted of a ring or mask (2) projected by light, prisms and/or semi-transparent mirror, and an apparatus for observing the image reflected by the surface, which allows the passage of light reflected off the patient's cornea through axes A and B, and indicates the eye to be measured, right or left.

The present invention refers to an ophthalmic device, for coupling in Slit Lamps or Ocular Microscopes, with the purpose of utilizing the image to selectively measure the cornea's curvature and examine its topography under the examination mode of the instrument in reference, which is composed by an illuminated ring or projection mask and a device to observe the reflected image from the surface where the ring is projected onto. The mask, coupled onto the objective lens or any fore part of the Slit Lamp, in projection axis A, allows the passage of light through axes A and B, and thus the reflected image from the patient's cornea or reflecting surface, through its central holes. The ring or mask allows the illuminated ring's or mask's image to be centered by the cornea or reflecting surface by a fixation point for the patient's eye, which must be aligned with the respective light ring and the system's optical axis. The alignment with the fixation point allows the passage of the ring's image reflected from the eye, through the center of the objective lens of the slit lamp, and by means of mechanical alignment of the patient's eye with the instrument's optical axis in such manner that the luminous device is positioned in the alignment axis with the eye. Such reflected image passes through axes A and B for reception, and the image is viewed through eyepiece lenses and/or screen (and/or observation device), respectively.

The invention, coupled to the Slit Lamp or an Ocular Microscope is composed of lenses, prisms, diaphragms and mirrors for focusing and receiving the reflected image, light signal emitters and receivers that emit and receive the reflection of the emitted signal, image sensors and a calculation system.

The instrument has two operational modes: the first mode measures corneal curvature radii in two orthogonal plans, while the second mode allows the measurement of corneal curvature radii in two non-orthogonal plans (bi-oblique astigmatism cases).

The two Slit Lamp eyepiece lenses are disposed on the alignment axis of the projection and imaging system.

The invention's projection system consists of a projected light ring onto the cornea in such manner that any distortion of the reflected image is analyzed for keratometric measurements.

The imaging system has an axis displaced from and substantially parallel to the aligning axis. This system deviates part of the image to the eyepieces of the slit lamp or ocular microscope and the other part to the image apparatus (or sensor) to be observed or displayed in a monitor, where both modes of operation of the instrument may be operated.

The calculation system consists in analyzing the deformation of the reflected image of the projected mire on the cornea, directly by observation or by computational methods, providing all parameters relatively to keratometry such as data and graphic interfaces of the examined cornea.

The invention, as claimed herein, especially developed for ophthalmic purposes, but with the possibility to measure curvature radii of any light reflecting surface, is composed of 5 fundamental parts: a) ring projection system; b) optics for viewing the examined eye; c) imaging system; d) control electronics; and e) mathematical and computerized system (images and data processing respectively), with the following scientific and construction concepts for industrial purposes.

The first part of the invention is the light ring projection system, which consists of a mire that is attached to a slit lamp or ocular microscope in order to obtain the reflected image of the said mire reflecting means for displacing an image from said aligning axis to an imaging system axis and for returning said image to said aligning axis in an erect and unreversed position in order to provide both mire and corneal images in vertical and horizontal alignment with the cornea itself.

The Slit Lamp is an integrating part of the optics for viewing the examined eye and for the optical imaging system, through its eyepiece lenses, for the viewing of reflected image from the cornea which the ring was projected onto, allowing, through an optical set, that includes objective lenses and an optical assembly for corneal measurement, the image to be seen by the equipment's eyepiece lenses placed on the alignment axis at the opposite end of the light device in such manner that the first and the second axis coincide for viewing through the imaging sensor simultaneously or not with the eyepiece lenses.

The imaging system, through the optical system coupled where the two axes meet, implemented in the microscope has the purpose of sending a percentage of the image obtained by the microscope to its eyepiece lenses and to the video or computer screen.

With respect to the invention's control electronics, they are characterized by a sensor that indicates the eye being measured, left or right, and controlling the projection ring as well as the image receiving and pausing system.

Finally, the mathematical or computerized system, deals with image processing and consists of observation, through direct or computerized methods, the reflected image from the cornea, distorted or not, and the calculations, which provides all keratometric data and data processing results of the cornea being examined.

### STATE OF THE ART

For many years, optometrists, ophthalmologists and researchers of the ocular area have used equipment to study the eye. Some of this equipment has been in use simply to observe parts of the eye, subjectively, such as: magnifying glasses, Slit Lamps and bio-microscopes. Other instruments referred to as keratometers or ophthalmology meters have been used to measure the radii of corneal curvature along the two main meridians of the eye. The functioning principle of such instruments is already well known, as shown by several chapters in books such as “The eye and Visual Optical Instruments” published in 1941 by Smith George and Atchison David A. Cambridge University Press, p. 175.

Currently, keratometers already carry technological advancements that allow the measuring of corneal curvature radii, some equipped with ring projection covering the entire surface, showing the topography of the cornea.

All of them have their own optics, some automated, but presenting high manufacturing costs for separate and exclusive optics for this kind of measuring.

The existing non-automated keratometers, besides giving a relatively broad scale of measure background, do not allow for non-orthogonal axes measurements (only orthogonal), possible only by automated equipment, greater part of which do not have measurement interval that is broad enough, in addition to extremely high cost.

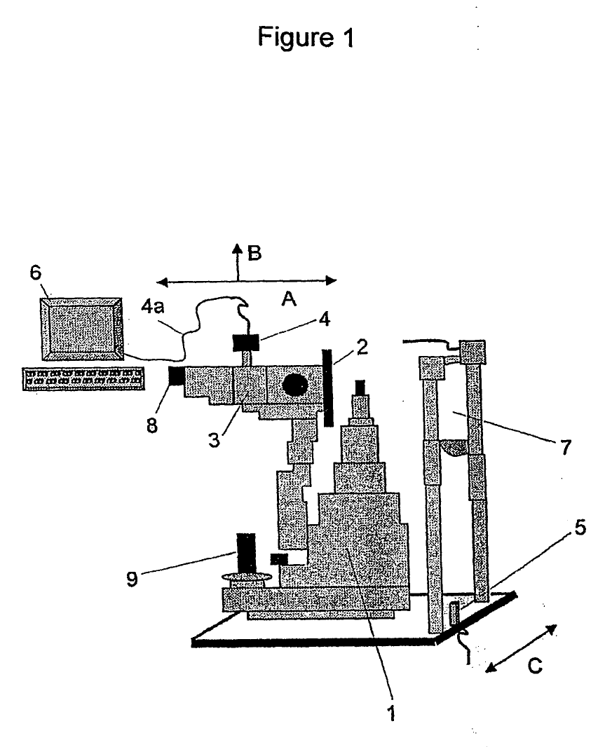

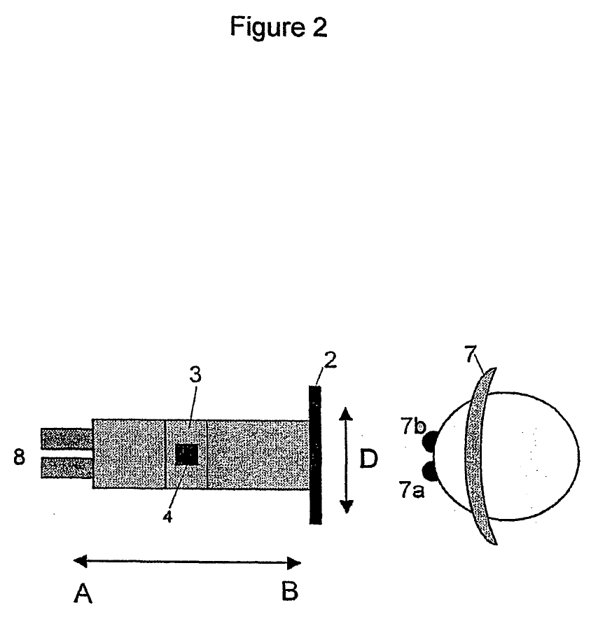

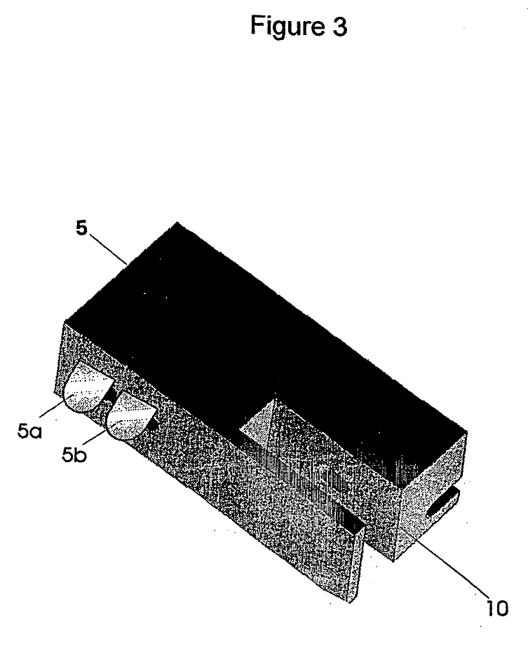

For better understanding and to show evidence of the invention's innovations, assisted through the drawings attached herein and illustrating the present request or patent, a detailed description of the keratometric module's frame is presented—FIG. 1—, constructed for coupling to instrument (1)—Slit Lamp or Microscope—with a projection illuminated ring (2)—FIG. 2—coupled to the objective lens or any fore part of the Slit Lamp (1), on projection axis A. Between the projection axis and reception axis A and the eyepiece lenses is inserted a system (3) for partial beam deviation to axis B, which is composed by prisms and/or semi-transparent mirror that deviates the reflected image of the ring's projection (2) onto the eye of the positioned patient (7) in axis A to the eyepiece lenses (8) of the instrument (1) and to axis B, at the imaging sensor (4) and further on to the observation apparatus (6)—video screen. The ring (2) is focused onto the patient's eye in position (7) by the joystick (9) of the instrument (1), which allows moving it in directions X, Y and Z of the Cartesian plane, and observed in real time in positions (8) and (6). The eye sensor (5), FIG. 3, placed in one of the instrument's (1) ends along axis C, has light signal emitters and receivers (5a) and (5b), and a hole for power supply cable, controlled by the microcomputer as shown in FIG. 3. The emitter (5a) emits a signal that reaches the instrument's assembly (1), which, if positioned in the same end as the emitter (5a) along axis C, reflects the signal transmitted by the emitter (5a), being received by the sensor (5b) whose reception indicates the instrument's (1) position and thus indicates the eye (7a), as shown by FIG. 2, being measured.

Absence of signal (5b), signal not reflected by instrument (1), indicates that the opposite eye is the one being measured.

To project a light ring (2) with the necessary luminosity on the patient's eye, a light ring or illuminated ring (2) is provided.

The light ring (2), better seen in FIG. 2, is opaque and has a central circular hole (11) with a light source, flashing or not, and two holes (12a and 12b) so that observation through the two Slit Lamp (1) eyepiece lenses is not blocked. There are several holes (13) placed in precise circular shape on the ring (2a), more than 5 in number, around the central hole (11). Several light sources (13a) are inserted into the holes (13), a source for each hole.

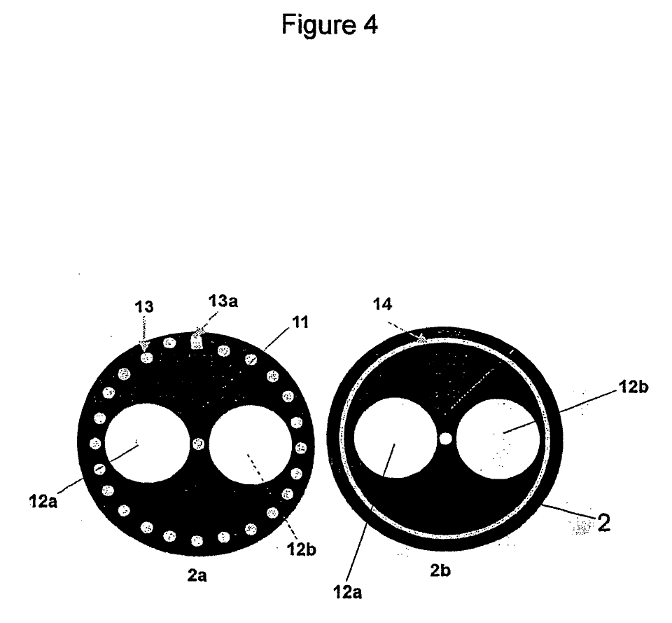

When the lights (13a) are activated, the light from these holes reach the patient's cornea (see FIG. 4) and project an image right behind the one reflected by a patient's eye (7a), the same occurring for the other eye (7b) when the equipment is positioned to examine it.

This reflected light is the image that goes though the instrument (1) under its measurement mode.

The apparatus for viewing the image is composed by a video sensor (4), information forwarding system to microcomputer (frame grabbers—boards or cable) (4a) and microcomputer screen (6) coupled to a microcomputer. Alternatively, the ring (2) may be like the other ring (2b) with an illuminated or luminous ring in precisely continuous circular shape.

As seen in FIGS. 1 to 4, an imaging system (3), (4), (4a) and (6) is placed along axis B coupled to the instrument (1) on its axis A.

The system uses the objective lens and image enhancing drum of the instrument (1) to optically obtain the image reflected by the patient's eye while the distorted or non-distorted reflection of the ring's projection (2) of a circular figure on the patient's eye is obtained and analyzed by a microcomputer. The distortion (deviation of the circularity) of reflected image of the projected light mire target is analyzed, and the dimension of the said image and quantity of distortion provides data for determining the radii of curvature (or refractive power) along the 360°, as well as the major and minor radii of curvature of the surface (or refractive power), which can be obtained in both modes of measurement (orthogonal or non-orthogonal).

Keratometry analysis is thus obtained.

FIG. 1, which accompanies and illustrates the invention shows the side view of the entire system of the present invention coupled to a Slit Lamp (A, B and C axes).

FIG. 2 presents the invention in frontal view for coupling to the present invention's system along axes A-B of FIG. 1.

FIG. 3 shows a topographic view of the frame, light signal emitters and receivers of the eye sensor used in the invention.

Finally, FIG. 4 shows the upper View of the ophthalmologic instrument developed for instrument 1, of ring projection 2 on the eyes 7a and 7b of the patient.

As known, an innovative process must incorporate industrial features and applications to satisfy cost/benefit needs of professionals of the area and patients, and stand out in the market.

The present invention has such requisites, with principles and singular characteristics, and which innovative method has proved fully efficient in its constitution and application according to the descriptive report. In view of such advantages, and the technical impact created, the present object gathers the necessary conditions to entitle it the privilege claimed.

1- “KERATOMETRIC MODULE FOR COUPLING TO SLIT LAMPS AND OR OCULAR MICROSCOPES”, characterized by an ophthalmic instrument constructed for coupling to instrument (1)—Slit Lamp or Ocular Microscope—which uses the image to selectively measure the curvature of the cornea and examine its surface under the examination mode of the instrument in reference, constituted of a ring (2) or mask projected by light, and by an observation device of the image reflected by the reflective surface. The ring or mask (2), coupled to the objective lens or any fore part of the Slit Lamp (1), on projection axis A, allows the passage of light through axes A and B, reflected on the patient's cornea or reflective surface, allowing the image of this ring or illuminated mask to be reflected by the cornea or reflective surface. A partial beam deviation system (3) is inserted between the projection axis and reception axis A on the eyepiece and axis B, and is composed by prisms and/or semi-transparent mirror that deviates the reflected image from the projection ring (2) on the positioned patient's eye (7) to axis A to the eyepiece lenses (8) of the instrument (1) and to axis B, to the image sensor (4) and observation apparatus (6)—video screen. The ring (2) is focused on the patient's eye in position (7) by the joystick (9) of the instrument (1), which allows moving it in directions X, Y and Z of the Cartesian plane, and be observed in real time at positions (8) and (6). An eye sensor (5) is placed at one of the instrument (1) ends along axis C, equipped with light signal emitters and receivers (5a) and (5b), and a hole for the power supply cables. The eye sensor (5) is controlled by the microcomputer. The emitter (5a) emits a signal that reaches the instrument's (1) frame, which, if positioned at the same end of the emitter (5a) along axis C, reflects the emitted signal by the respective emitter (5a) and is received by the sensor (5b) whose signal reception indicates the position of the instrument (1) and thus indicates the eye (7a) that is being measured. The absence of signal in sensor (5b),—signal not reflected by the instrument (1)—indicates that the opposite eye is the one being measured.

2- “KERATOMETRIC MODULE FOR COUPLING TO SLIT LAMPS AND OR OCULAR MICROSCOPES”, according to claim 1, it is characterized by a light ring (2) that has a central circular aperture (11) flashing or not, two holes (12a and 12b) for allowing observation through two eyepiece lenses of the Slit Lamp (1) and several holes (13), more than 5 in number, placed in precise circular shape, around the central aperture (11). Various light sources (13a) area inserted in the holes (13), one source for each hole, and when the lights are activated, the light coming from these holes reach the patient's cornea, projecting an image right behind it, which is reflected by one of the patient's eyes (7a), the same occurring for the other eye (7b) when the equipment is positioned for it, being that this reflected light is the image that passes through the instrument (1) and reaches the image viewing apparatus composed by a video sensor (4), information forwarding system to microcomputer (video transmission board or cable) (4a) and micro computer screen (6) coupled to the micro computer, and alternatively, a ring (2) being like a light ring 2b (FIG. 2), which is a luminous ring in precise continuous circular shape.
